(19) United States
(12) Patent Application Publication
(10) Pub. No.: US 2007/0134753 A1
(43) Pub. Date: Jun. 14, 2007

(54) Assay for determining the activity of fatty acid amide hydrolase

(76) Inventors: Ann Johanna Barbier, San Diego, CA (US); Sandy J. Wilson, San Diego, CA (US); Curt Mazur, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/531,777

(22) PCT Filed: Oct. 21, 2003
(86) PCT No.: PCT/US03/33354

§ 371 Date: Apr. 19, 2005

Related U.S. Application Data
(60) Provisional application No. 60/420,065, filed on Oct. 21, 2002.

(51) Int. Cl. C12Q 1/34

(52) U.S. Cl. 435/18

(57) ABSTRACT

The invention provides methods of assaying fatty acid amide hydrolase (FAAH) activity adaptable for high through-put screening. The methods provide for separating a labeled substrate from at least one labeled hydrolysis product, the separation facilitating the quantification. The invention also provides methods of identifying a compound to be tested as an inhibitor or an activator of FAAH activity through the addition of the compound to be tested to a reaction mixture and comparison of the enzyme activity in the presence and absence of the compounds to be tested. The methods are adaptable for use in detecting altered FAAH activity in patients, for example, those at risk for in vitro fertilization failure, or at risk for, or suffering, addictions.

### FIELD OF THE INVENTION

The present invention relates to methods for determining the activity of and identifying modulators of fatty acid amide hydrolases. More specifically, the invention relates to an assay, adaptable for high throughput screening, for compounds that alter fatty acid amide hydrolase activity.

### BACKGROUND

The identification of anandamide (N-arachidonoylethanolamine, AEA) as an endogenous ligand for the cannabinoid 1 receptor (Devane et al. (1992) Science 258:1946-1949) evoked much scientific interest in the function of bioactive lipids. Other examples of endogenous ligands are oleamide (cis-9,10-octadecenoamide), best known for its sleep-inducing properties (Cravatt et al. (1995) Science 268:1506-1509), and 2-arachidonoylglycerol, reported to be neuroprotective after brain injury (Panikashvili et al. (2001) Nature 413:527-531).

The main mechanism for the termination of the biological activity of anandamide is hydrolysis (Giuffrida et al. (2001) J. Pharmacol Expt. Ther. 298:7-14); the existence of an anandamide transporter has also been proposed (Compton and Martin (1997) J. Pharmacol. Expt. Ther. 263:1138-1143).

The enzyme responsible for the hydrolysis of anandamide, oleamide and 2-arachidonoylglycerol was cloned in 1996 and named fatty acid amide hydrolase, or FAAH (Cravatt et al. (1996) Nature 384:87-87). FAAH (EC 3.5.1.4) is a membrane-bound enzyme with broad substrate specificity which is expressed in a wide variety of human tissues and cell lines (for review see Ueda et al, Chem. Phys. Lipids. 108: 107-121, (2000); Fowler et al., Biochem. Pharmacol. 62: 517-526, (2001)).

Inhibitors of FAAH have been predicted to potentiate the effects of the endogenous cannabinoids and thereby promote sleep, muscle relaxation and analgesia (Fowler et al. (2001) Biochem. Pharmacol. 62:517-526). Efforts to identify useful inhibitors have been hampered by the lack of simple, reproducible assays suitable for high-throughput screening. Published methods include reversed phase HPLC (9) and thin-layer chromatography (Deutsch and Chin (1993) Biochem: Pharmacol. 46:791-796). A fluorescence displacement method has also been described (Thumser et al. (1997) Biochem. Pharmacol. 53:433-437). An additional assay relies on extraction of the hydrolysis product with a chloroform: methanol mixture (Maurelli et al. (1995) FEBS Lett. 377:82-86), followed by counting of radioactivity. However, the toxicity of the chloroform and the cumbersome physical manipulations of this method preclude the adaptation of this assay to a high-throughput format

There is a need in the art, therefore, for improved FAAH assays, particularly those which can be adapted to high throughput screening, for determining the activity of FAAH and for identifying modulators of FAAH.

### SUMMARY OF THE INVENTION

In one aspect, the instant invention provides methods for assaying the activity and amount of fatty acid amide hydrolase (FAAH) based on differences in the physicochemical and binding properties of a FAAH substrate, and the products of its hydrolysis. For example, anandamide is hydrolyzed by FAAH to arachidonic acid and ethanolamine (FIG. 1). In brief, a substrate, for example ³H-anandamide (ethanolamine 1-³H), is incubated with a putative source of FAAH activity in a reaction mixture. The FAAH activity, where present, catalyzes the hydrolysis of the substrate to form at least one radiolabeled hydrolysis product; the example substrate ³H-anandamide is converted to labeled ethanolamine and unlabeled arachidonic acid. This labeled product and the labeled substrate are separated from each other and the loss of labeled substrate, or preferably, the formation of labeled product, is measured. In certain preferred embodiments, the assays are performed in parallel or in sets wherein assays conducted in the presence of compounds to be tested for their ability to modulate the FAAH activity are compared with those conducted in the absence of the compounds to be tested. In other preferred embodiments samples from patients can be assayed to determine if the FAAH activity is altered relative to a predetermined activity value.

In one aspect, the invention provides improved methods of measuring fatty acid amide hydrolase activity comprising combining a sample suspected of containing fatty acid amide hydrolase, with a labeled substrate of fatty acid amide hydrolase to form a reaction mixture; incubating the reaction mixture under conditions which allow the fatty acid amide hydrolase to hydrolyze the labeled substrate, thereby forming at least one labeled hydrolysis product; contacting the incubated reaction mixture with a selective binding material wherein the selective binding material binds either the labeled substrate or the labeled product, but not both, thereby forming a bound labeled complex; separating the bound labeled complex from the unbound labeled compound, thereby effectuating a separation of the labeled substrate from labeled product; and determining the amount of labeled substrate hydrolyzed or the amount of labeled hydrolysis product formed; thereby indicating the fatty acid amide hydrolase activity of the sample.

In another aspect of the invention, methods are provided for identifying compounds which can modulate the activity of a FAAH enzyme. The methods comprise the steps of comparing the activity of a FAAH as assayed by the above method in the presence and in the absence of a test compound added to the reaction mixture; wherein a change in the activity of the fatty acid amide hydrolase indicates that the test compound modulates the activity of the fatty acid amide hydrolase. In various embodiments, the methods can be used to identify useful inhibitors or enhancers of FAAH activity.

The assay methods provided herein are adaptable for use in high throughput screening systems and are contemplated to be used in drug discovery efforts. High throughput screening using the assay methods of the present invention will allow libraries of test compounds (for example, libraries produced by techniques of combinatorial chemistry) to be used in rational screening programs to identify inhibitors and enhancers of FAAH activity which are useful as candidates for drugs

In another aspect, the invention provides methods of determining altered FAAH activity in a patient. The methods comprise the steps of obtaining a sample containing cells from the patient; lysing the cells to form a cell lysate; combining the cell lysate with a labeled substrate of the fatty acid amide hydrolase, to form a reaction mixture; incubating the reaction mixture under conditions sufficient to allow a fatty acid amide hydrolase present in the cell lysate to hydrolyze the labeled substrate, thereby forming at least one labeled hydrolysis product; contacting the incubated reaction mixture with a selective binding material; wherein the selective binding material binds either the labeled substrate or a labeled hydrolysis product, but not both, thereby forming a bound labeled complex; separating the bound labeled complex from the unbound labeled compound, thereby effectuating a separation of the labeled substrate from labeled product; determining an amount of labeled substrate hydrolyzed, or labeled hydrolysis product formed, thereby indicating the fatty acid amide hydrolase activity of the sample; and comparing the activity of the sample from the patient with a predetermined value for activity, to determine if the patient has altered fatty acid amide hydrolase activity relative to the predetermined value for activity.

These and other aspects of the present invention will be described in further detail in the Detailed Description set forth below.

### BRIEF DESCRIPTION OF THE FIGURES

FIG. 1. Schematic diagram of a FAAH assay. The FAAH activity in T84 membranes converts anandamide [1-³H-ethanolamine] into radiolabeled ethanolamine and unlabeled arachidonic acid. The labeled anandamide and unlabeled arachidonic acid are selectively bound to charcoal in filterplates, whereas the radiolabeled ethanolamine is collected in the flow-through and counted.

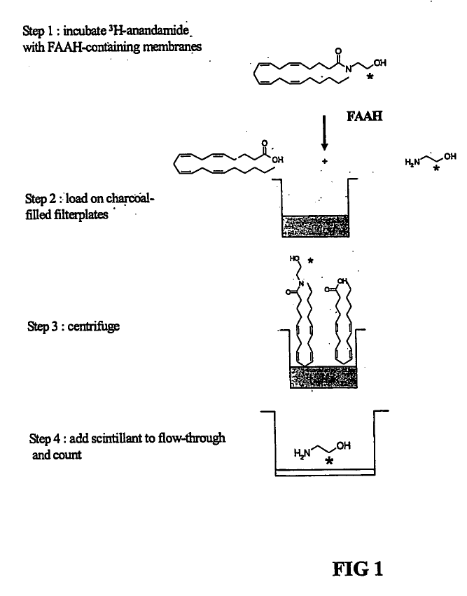

FIG. 2 depicts an embodiment of a FAAH assay method. The reaction takes place in a reaction plate at room temperature. After 60 minutes, 60 μl of the reaction mixture is transferred to a charcoal-filled filter plate. The filter plate is fitted on top of a Dynex plate; the assembly is then centrifuged for 5 minutes at 2000 rpm.

FIG. 3. Arachidonic acid and anandamide, but not ethanolamine, bind to activated charcoal. Four different radiolabeled tracers were used: anandamide [1-³H-ethanolamine], anandamide [arachidonyl-5,6,8,9,11,12,14,15-³H], arachidonic acid [5,6,8,9,11,12,14,15-³H (N)] and ethanolamine ([2-¹⁴C] ethan-1-ol-2-amine hydrochloride). The tracers were incubated with membranes prepared from mouse liver, T84 cells, HeLa cells or with vehicle, for 60 minutes at room temperature. The percentage (calculated from the average of triplicate determinations±Standard Error of the mean (s.e.m.)) of total radioactivity recovered in the flow-through is shown.

FIGS. 4A-4B. Characterization of a preferred assay. A: Time-course of hydrolysis of ³H-anandamide by FAAH at room temperature and at 37° C. The average of triplicate determinations±s.e.m. is shown. B: Determination of Kₘ for FAAH. T84 cell membranes (3.5 μg protein per well) were incubated with a range of concentrations of ³H-anandamide. The reactions were carried out at room temperature in the presence or absence of inhibitors (either oleyl trimethylfluoro ketone (OTFMK) or methyl arachidonyl fluorophosphate (MAFP)). The experiment where oleyl trimethylfluoro ketone was used to determine nonspecific binding is shown, the results with MAFP were comparable. Each point reflects the average of triplicate determinations±s.e.m.

### DETAILED DESCRIPTION OF THE INVENTION

The reference works, patents, patent applications, and scientific literature, including sequences denoted by their accession numbers (e.g. accession numbers to GenBank database sequences), that are referred to herein are hereby incorporated by reference in their entireties.

Various definitions are used throughout this document. Most words have the meaning that would be attributed to those words by one skilled in the art. Words specifically defined either below or elsewhere in this document have the meaning provided in the context of the present invention as a whole, and as are typically understood by those skilled in the art. Where possible undefined words should be understood to have the usual meaning to the skilled artisan. Any conflict however between an art-understood definition of a word or phrase and a definition of the word or phrase as specifically taught in this specification shall be resolved in favor of the latter.

Standard reference works setting forth the general principles of recombinant DNA technology known to those of skill in the art include Ausubel et al., Current Protocols in Molecular Biology, John Wiley & Sons, New York (2002); Sambrook et al., Molecular Cloning: A Laboratory Manual, 2d Ed., Cold Spring Harbor Laboratory Press, Plainview, N.Y. (2001); Kaufman et al., Eds., Handbook of Molecular and Cellular Methods in Biology and Medicine, CRC Press, Boca Raton (1995).

As used herein, a “label” is a composition detectable by spectroscopic, photochemical, biochemical, immunochemical, or chemical means. For example, useful labels include radioisotopes and fluorescent labels. Examples of radioisotopes that may be used in the method of the invention include ³H and ¹⁴C.

As used herein, “purified” refers to at least partial separation of a molecule from other molecules with which it is normally associated. For example, a purified protein is a protein that is at least partially separated from other cellular material with which it is normally associated.

As used herein, the term “murine” means originating in a member of the family Muridae. A murine FAAH preferably originates in a mouse, or rat.

In a first aspect the invention provides methods of assaying a fatty acid amide hydrolase (FAAH). In a presently preferred embodiment, the method of assay comprises the steps of combining a sample suspected of containing a FAAH with a labeled substrate of FAAH to form a reaction; incubating the reaction mixture under conditions sufficient to allow the fatty acid amide hydrolase to hydrolyze the labeled substrate, forming one or more labeled hydrolysis products; contacting the incubated reaction mixture with a selective binding material; wherein the selective binding material binds either the labeled substrate or a labeled hydrolysis product, but not both, to form a bound labeled complex; separating the bound labeled complex from the unbound labeled compound, thereby effectuating a separation of the labeled substrate from labeled product; and determining an amount of labeled substrate hydrolyzed, or labeled hydrolysis product formed, thereby indicating the fatty acid amide hydrolase activity of the sample.

The sample, in preferred embodiments, is a biological sample; or a sample comprising biological material, in particular biological membranes. In other preferred embodiments, the sample is from a purification step in the purification of FAAH from a biological source. The sample comprises biological membranes or portion thereof, or comprises lipid bilayers, or artificial membrane systems, monolayers, vesicles or micelles. A sample in some embodiments comprises a purified or recombinant FAAH reconstituted into a phospholipid-containing reaction mixture.

The substrate may be any substrate, putative substrate, or substrate analog of FAAH. The assay is also adapted for use to determine the utility of a compound as a substrate of FAAH. Substrates preferred for use in various embodiments of the present invention include, but are not limited to endocannabinoids or analogs thereof, fatty acid ethanolamides or analogs thereof, fatty acid primary amides or analogs thereof, and analogs of any of the foregoing labeled with a detectable label. Presently preferred substrates of particular interest include, for example, anandamide, oleamide, and 2-arachidonoylglycerol.

The substrate may be radioisotopically labeled in any manner known in the art for labeling compounds for detection. Presently preferred isotopes, such as ³H or ¹⁴C, are readily detected via liquid scintillation counting and can facilitate adaptation of the assays for high throughput drug screening. Synthetic substrates so labeled are readily available commercially or can be synthesized.

Other labels such as fluorescent labels capable of detection, for example fluorimetric detection, are also readily adapted to high throughput screening. Preferred fluorescent labels are detectable in biological systems with both proteins and nucleic acids present, therefore preferred labels when assaying cruder cell lysates and homogenates possess both excitation and emission optima which are not masked by these other biological components. In one embodiment the unhydrolyzed labeled substrate has identical fluorescent properties with the hydrolysis product. It is also possible to design substrates wherein hydrolysis results in a change in fluorescence, for example, where a dye-dye interaction in the unhydrolyzed molecule is required to maintain a ground state. Such substrates are useful in conjunction with the present invention. Colorimetric labels are also contemplated for use herein. Generally, appropriate labels are those which do not alter the substrate's susceptibility to hydrolysis by the enzyme and for which detection systems have been developed.

Preferably the substrate is present at concentrations at vast excess relative to the concentration of the enzyme. Under such conditions classical enzyme kinetics can be used to determine a rate constant (Kₘ) and a velocity (Vmax) of the reaction. Kinetic studies are useful for mechanistic studies; they are also powerful tools for evaluating inhibitors (see below). Standard texts directed at those skilled in the art of enzyme kinetics such as Segel, I. H., Enzyme Kinetics: Behavior and Analysis of Rapid Equilibriumn and Steady-State Enzyme Systems (1993, Wiley-Interscience, ISBN 0471303097) provide complete guidance to establishing these parameters and demonstrate the use, for example, of Lineweaver-Burke plots as graphical tools to simplify the determination of the kinetic parameters.

High throughput screening systems are known in the art. Such systems often involve the use of multiwell plates to increase the number of assays conducted simultaneously from a few to a hundred, a few hundred or even a few thousand. Presently preferred high throughput screening adaptions of the methods of the present invention provide capability of screening about one hundred to about one or more thousand assays in a short time. Presently preferred high throughput screening systems include robotic components for example for sample handling, dispensing, reagent addition, and other functions to improve accuracy and eliminate the labor intensive aspects of large numbers of assays. Detection systems for high throughput screening programs are known to those of ordinary skill in the art. Preferred detection systems include, but are not limited to, scintillation counting (including, for example solid and liquid scintillation for counting gamma or beta particles, or luminescent samples, filter counting, Cerenkov counting, and scintillating microplate counting), fluorescence detection (including, for example, intensity, fluorescence polarization, time-resolved fluorescence, fluorescence resonance energy transfer (FRET)), luminescence, and absorbance.

In a presently preferred embodiment, to achieve high-throughput screening, samples are placed on a multicontainer carrier or platform. A multicontainer carrier facilitates measuring reactions of a plurality of candidate compounds simultaneously. For illustration purposes, but not by way of limitation, a multi-well microplate, such as a 96 or a 384 well microplate, that can accommodate 96 or 384 different test reactions, is used as the carrier. Such multi-well microplates, and methods for their use in numerous assays, are both known in the art and commercially available through sources such as Sigma Chemical Co., BIOCHEMICAL ORGANIC COMPOUND AND DIAGNOSTIC REAGENTS, 2002, pages 2495-2511.

The methods of the present invention are adaptable to miniaturization techniques. Assays for purposes of high throughput screening are often conducted in small volumes. Procedures are currently known to those of skill in the art of reducing volumes of assays in a variety of ways. Microfluidics and microcapillary methodologies now enable assays to be performed down to nanoliter quantities. It is to be understood that the basic principles of the methods and assays apply notwithstanding the volume of the assay, or the manner in which the materials are measured or transported.

The process of determining incubation conditions is routine in the art Preferred incubation conditions relate, for example, to enzyme stability. Temperature optimums are routine to determine given the specification of the assay and should be determined for a given enzyme and substrate combination for optimum results. Times and other conditions for incubation involve likewise routine determinations. Preference is given to those conditions which result in linearity of the assay. Presently preferred temperatures and times include room temperature for 1 hour, or 37° C. for 30 min, using FAAH from a variety of sources and using anandamide as a substrate. Standard texts directed at those skilled in the art of enzyme assays such as Segel, supra, provide complete guidance to optimizing assays, including the incubation conditions. Further characterization of the preferred embodiments is provided in the working examples.

The methods also comprise a step wherein the incubated reaction mixture is contacted with a selective binding material. It is important to note that the selective binding material preferred for this assay may vary with the substrate selected. The selective binding materials comprise materials which can readily be separated from the bulk reaction mixture by separation techniques which are known in the art. Effective separations can be based on differences in particle size, density, composition and magnetic susceptibility. Material is separated from a reaction mixture by, for example, gravity settling, filtration (including, for example, membrane separations), centrifugation, A presently preferred method of contacting the incubated reaction mixture with a selective binding material comprises activated charcoal.

Where the substrate is anandamide, for example, activated charcoal binds, through adsorption, the substrate and one of the hydrolysis products, arachidonic acid. The other hydrolysis product, ethanolamine, does not adsorb to the activated charcoal. For preferred embodiments, the anandamide substrate is labeled on the ethanolamine moiety. The FAAH hydrolyzes the labeled anandamide into labeled ethanolamine and unlabeled arachidonic acid. After adsorption of the labeled substrate and the unlabeled arachidonic acid to the charcoal, the labeled ethanolamine in solution can be separated from the bound labeled substrate through a simple filtration step. This facilitates the measurement of product without interference from substrate. By removing some compounds through the binding process, the sample is less likely to interfere, for example by quenching, with the measurement of the labeled product. Existing technology has already been adapted for filtering large numbers of samples simultaneously, for example in multiwell filters. This attribute also is adaptable therefore to high throughput screening programs.

Although it is often preferred to measure the formation of product in the methods of this invention, assay conditions and selective binding material may be selected wherein the disappearance of substrate is measured. This indirect method is adaptable particularly where a partially purified or substantially purified enzyme is used, or where it is known that there is only one route by which substrate disappears from the reaction mixture. It is also preferred to measure substrate disappearance wherein a selective binding material which binds the substrate is not readily available. This can be useful in particular in embodiments where the substrate cannot practicably be bound to effect its separation from free hydrolysis product but where the hydrolysis product can be more readily bound to effectively separate it from the free labeled substrate. This gives a practitioner the flexibility to choose from a broader range of selective binding materials.

The determination of the amount of labeled substrate hydrolyzed or the labeled hydrolysis product formed is preferably a direct method of quantitating the amount of label present. As discussed above, a variety of detection techniques are suitable for the present invention, and an appropriate detection method relates to the properties of the label present on the substrate or the product. Presently preferred for use with the methods of the invention are radioisotopically labeled substrates and fluorescently labeled substrates, quantified respectively by scintillation counting and fluorescence detection, respectively. In one preferred embodiment, the substrate is ³H-anandamide, the detection is of the product, ³H-ethanolamine, formed, and the detection is with a liquid scintillation counting plate reader from the filtrate of an assay conducted in a multiwell plate.

In another aspect of the invention, methods are provided for identifying modulators of fatty acid amide hydrolase. The methods comprise comparing the activity of fatty acid amide hydrolase as assayed by the method as described herein above, in the presence and in the absence of a test compound added to the reaction mixture; a change in the activity of the fatty acid amide hydrolase indicates that the test compound modulates the activity of the fatty acid amide hydrolase.

In preferred embodiments, the modulator to be identified is part of compound library, for example, a library of compounds formed by combinatorial chemistry, or a library of related compounds identified of synthesized as part of a research program. The putative modulators (i.e the compounds to be tested, test compounds) are preferably a small molecule with properties that would allow it to have utility for pharmaceutical compositions. Preferred modulators have low nonspecific toxicity, and high specificity for modulating FAAH.

Modulators include inhibitors of FAAH activity and activators of FAAH. Assay parameters such as concentration of substrate, incubation conditions, and concentration of compounds to be tested may be varied to more fully appreciate the modulation effects of a test compound.

Preferred modulators that are inhibitors can be identified by their ability to decrease the activity of FAAH relative to that of a control reaction mixture lacking the test compound. Preferably, reaction volumes are maintained constant by adjusting the volume to compensate for the addition of a test compound where the addition of a test compound alters the reaction volume. Most preferably any difference in volume is compensated for by a corresponding addition of a volume, equal to the difference, of the vehicle in which the test compounds are dissolved.

In one aspect of the invention, the modulator identified is an inhibitor of the FAAH activity. Enzyme inhibitors include reversible and irreversible inhibitors. Preferred inhibitors in some embodiments are reversible inhibitors. Reversible inhibitors of FAAH include competitive inhibitors, which raise the apparent Kₘ of the reaction, noncompetitive inhibitors, which reduce the Vₘₐₓ of the enzyme, and inhibitors which affect both Kₘ and Vₘₐₓ, namely, mixed inhibitors and uncompetitive inhibitors. The type of inhibitor identified in screens can be determined through the use of kinetic assay studies according to the methods of the present invention. For the purposes of the present invention, however, any compound which decreases the apparent activity of the enzyme is considered a modulator of the inhibitor type. In other preferred embodiments inhibiting modulators include irreversible inhibitors as well as noncompetitive inhibitors, whose action may be irreversible.

In preferred embodiments the test compound inhibits the FAAH reproducibly, as determined by a statistically significant difference by an appropriate statistical test. Such tests are known to those of skill in the art of statistical determinations in scientific measurements, such as those of biological or biochemical systems. In some embodiments, the test compound inhibits fatty acid amide hydrolase activity about 1%, or at least about 2%, 3%, 4%,5%, 10%, 20%, 30%, 40%, 50%, 60%, 70%, 80%, or 90%, or about 95% or more, or up to 100%. In other preferred embodiments, the inhibitor statistically significantly increases the apparent Kₘ or decreases the Vₘₐₓ of the reaction.

In another aspect of the invention, the test compound increases fatty acid amide hydrolase activity. Various types of enzyme activators are known to those of skill in the art. Some activators are known to activate enzyme reactions by increasing the velocity of the reaction, others are known to alter the equilibrium attained. Activators that combine reversibly with enzyme reaction components (for example, the enzyme, the substrate, or enzyme-substrate or enzyme-product intermediates) to increase the velocity of the reaction are presently preferred. Nonspecific activators may also increase the reaction rate. For purposes of the present invention, any compound that increases the apparent activity of the enzyme is considered a modulator of the activator type. For example many enzymes are known to have increased activity in the presence of certain anions. Other enzymes, in particular those acting on water-insoluble and charge-neutral molecules, are activated by negatively charged lipophilic molecules. Some membrane bound enzymes are known to be activated, for example, by specific phospholipids.

In preferred embodiments the test compound activates the FAAH reproducibly, as determined by a statistically significant difference by an appropriate statistical test. Such tests are known to those of skill in the art of statistical determinations in scientific measurements, such as those of biological or biochemical systems. In some embodiments, fatty acid amide hydrolase activity is increased about 1%, or at least about 2%, 3%, 4%, 5%, 10%, 20%, 30%, 40%, 50%, 60%, 70%, 80%, or 90%, or about 95% or more, or up to 100%. In other embodiments, activation is 10-30%, 30-50%, 50-70%, 70-90%, 90-110%. In some embodiments, the activator increases FAAH activity 100-300%, 300-500%, 500-700%, or 700-900% or more.

In certain preferred embodiments of the methods of the invention, the fatty acid amide hydrolase is a crude cell lysate or a cell homogenate. In some embodiments the FAAH is isolated partially or substantially from cells. In other embodiments, the fatty acid amide hydrolase is recombinantly produced. The fatty acid amide hydrolase may be from any biological source including mammalian fatty acid amide hydrolases from such animals as pigs, rodents (including rats and mice), and humans, or it may be recombinant FAAH produced in any organism known to be useful for the production of recombinant proteins. Synthetic FAAH based on a particular known amino acid sequence, or on a consensus or combination of any number of sequences that result in an active FAAH is also contemplated for use herein.

Examples of known amino acid sequences of fatty acid amide hydrolases from various biological sources include, for example, SEQ ID NOS: 2, 4, 6, and 8. Examples of known nucleic acid sequences encoding fatty acid amide hydrolases include SEQ ID NOS:1, 3, 5 and 7. Persons of ordinary skill in the art will recognize that such sequences and the modifications thereof which retain activity, can be used in accordance with the techniques found in references such as those provided above to generate, for example, biological FAAH, recombinant FAAH, overproduced FAAH, or genetically modified FAAH. It is contemplated herein that any form of FAAH is adapted for assay with the methods of the present invention.

In a preferred embodiment the assay is used to measure the activity and identify modulators of FAAH with altered amino acid sequences. Such altered FAAH can result from mutations in the genes which encode FAAH enzymes. In one embodiment, the assay is used to study the activity of, and identify modulators of, altered FAAH in connection with cannabis abuse. For example, a recent paper by Sipe et al. (PNAS 99:8394-8398, 2002) describes a strong association between the substitution of Thr for Pro at position 129, and substance abuse in humans. The use of the assay of the present invention may help identify FAAH from humans with higher specific activities or higher levels of net activity, thereby resulting in reduced concentrations of endocannabinoids in the brain, thereby leading to a tendency to seek external cannabinoids to compensate.

In another aspect, the invention provides methods of determining altered FAAH activity in a patient. The methods comprise the steps of obtaining a sample containing cells from the patient; lysing the cells to form a cell lysate; combining the cell lysate with a labeled substrate of the fatty acid amide hydrolase, to form a reaction mixture; incubating the reaction mixture under conditions sufficient to allow a fatty acid amide hydrolase present in the cell lysate to hydrolyze the labeled substrate, thereby forming at least one labeled hydrolysis product; contacting the incubated reaction mixture with a selective binding material; wherein the selective binding material binds either the labeled substrate or a labeled hydrolysis product, but not both, thereby forming a bound labeled complex; separating the bound labeled complex from the unbound labeled compound, thereby effectuating a separation of the labeled substrate from labeled product; determining an amount of labeled substrate hydrolyzed, or labeled hydrolysis product formed, thereby indicating the fatty acid amide hydrolase activity of the sample; and comparing the activity of the sample from the patient with a predetermined value for activity, to determine if the patient has altered fatty acid amide hydrolase activity relative to the predetermined value for activity.

Preferably, the fatty acid amide hydrolase activity from a sample of fluid or tissue originating from the patient is measured. Presently preferred are blood, lymph or tissue samples. In particularly preferred embodiments, the sample is from lymphocytes from the patient. In preferred embodiments, where the sample is from a woman, the results enable a physician to screen pregnant women for risk of miscarriage (spontaneous abortion) or to screen women seeking fertility treatment for risk of failure of in vitro fertilization procedures. In preferred embodiments, results from the patient's sample are compared to results from samples of normal individuals, particularly those comparably positioned in terms of demographic criteria. Results of the assays of the present method may be used to determine either or both of altered specific activity (FAAH activity per unit of FAAH mass) or altered total FAAH activity (net amount of product formed per time). For example, increases in activity can be due to increases in specific activity of the FAAH, for example by alteration of the active site via amino acid alteration, gene mutation and the like, or through alteration of the total amount of FAAH protein present, without a change in the specific activity.

In the methods of the invention, the fatty acid amide hydrolase may be substantially purified from the lymphocyte cell lysate. FAAH may be derived from any source, such that the FAAH retains the potential for FAAH activity. For example, FAAH may be purified from cells expressing FAAH, FAAH may be produced in a recombinant system, including, but not limited to bacterial cells, yeast cells, insect cells, and mammalian cells. FAAH may be derived from any organism that produces FAAH; For example, FAAH may be derived from mammalian cells such as human cells, rodent cells (e.g., mouse and rat cells), or from any other organism that produces FAAH that has FAAH activity. Recombinant FAAH may be produced using techniques known in the art with any nucleic acid sequence encoding FAAH. Examples of cloned FAAHs include various mammalian FAAH such as, pig FAAH, rodent FAAH (e.g., mouse FAAH and rat FAAH), and human FAAH. DNA sequences encoding FAAH and polypeptide sequences of various FAAHs are included in the appended Sequence Listing.

FAAH may be purified by standard methods in the art. The FAAH preparation may be in association with cell membranes or artificial membranes, or may be substantially free of cellular material.

Throughout the specification, reference is made to certain publications and patents. The entireties of each of these references is incorporated herein and forms a part of this disclosure.

### EXAMPLES

### Example 1

Radiolabeled anandamide [1-³H-ethanolamine] was obtained from American Radiolabeled Chemicals (10-20 Ci/mmol, catalog number ARC-626; St. Louis, Mo., USA). Anandamide [arachidonyl-5,6,8,9,11,12,14,15-³H] was obtained from Perkin Elmer (160-240 Ci/mmol, catalog number NET-1073, Boston, Mass., USA), as was radiolabeled arachidonic acid-[5,6,8,9,11,12,14,15-³H (N)] (180-240 Ci/mmol, catalog number NET298Z). Radiolabeled ethanolamine ([2-¹⁴C]Ethan-1-ol-2-amine hydrochloride) was purchased from Amersham Pharmacia Biotech (55 mCi/mmol, catalog number CFA329). Methyl arachidonyl fluorophosphate (MAFP) and oleyl trifluoromethyl ketone (OTFMK) were obtained from Cayman Chemical, (Catalog number 70660 and 6260, respectively Ann Arbor, Mich., USA). Activated charcoal was from Aldrich Chemical, Milwaukee, USA. Arachidonic acid, anandamide, oleic acid, oleamide, phenylmethylsulfonyl fluoride and CuSO₄ were from Sigma, St. Louis, Mo., USA.

T84 human colorectal carcinoma cells (catalog number CCL-248, American Tissue Culture Collection, Manassas, Va., USA) were identified as expressing human FAAH based on gene expression profiling of cell lines using DNA microchips (not shown). The cells were cultured in a 1:1 mixture of Ham's F-12 media and Dulbecco's Modified Eagle Medium (Invitrogen) with 5% fetal bovine serum (HyClone), 50 U/ml penicillin and 50 μg/ml streptomycin (Invitrogen). The cells were harvested by scraping into PBS and pelleted in a clinical centrifuge at 1000 rpm. The resulting pellets were then stored at −80° C. until needed.

T84 frozen pellets were homogenized in FAAH assay buffer (125 mM Tris, 1 mM EDTA, 0.2% Glycerol, 0.02% Triton X-100, 0.4 mM Hepes, pH 9) (Boger et al. (2000) Proc. Natl. Acad. Sci. USA 97:5044-5049) and diluted to a final protein concentration of 70 μg/ml. Unless otherwise indicated, the assay mixture consisted of 50 μl of the cell homogenate, 10 μl of the appropriate inhibitor, and 40 μl of 40 nM ³H-AEA, added last, for a final tracer concentration of 16 nM. The reactions were originally done at 37° C. for 30 min; but subsequent experiments indicated that the enzyme displayed good activity at room temperature, and experiments were performed at room temperature for 60 minutes unless otherwise indicated.

During the one hour incubation, 96-well Multiscreen filter plates (catalog number MAFCNOB50; Millipore, Bedford, Mass., USA) were loaded with 25 μl activated charcoal (Multiscreen column loader, catalog number MACL09625, Millipore) and washed once with 100 μl methanol. Also during the incubation, 96-well DYNEX MicroLite plates (catalog number NL510410) were loaded with 100 μl MicroScint40 (catalog number 6013641, Packard Bioscience, Meriden, Conn., USA). After the one hour incubation, 60 μl of the reaction mix was transferred to the charcoal plates, which were then assembled on top of the DYNEX plates using Centrifuge Alignment Frames (catalog number MACF09604, Millipore). The unbound labeled ethanolamine was centrifuged through to the bottom plate (5 min at 2,000 rpm), which was preloaded with the scintillant, as described above. The plates were sealed and left at room temperature for 1 hour before counting on a Hewlett Packard TopCount. For determination of Kₘ values, 1 μM ³H-AEA was combined with 30 μM unlabeled AEA and serial 2-fold dilutions were made.

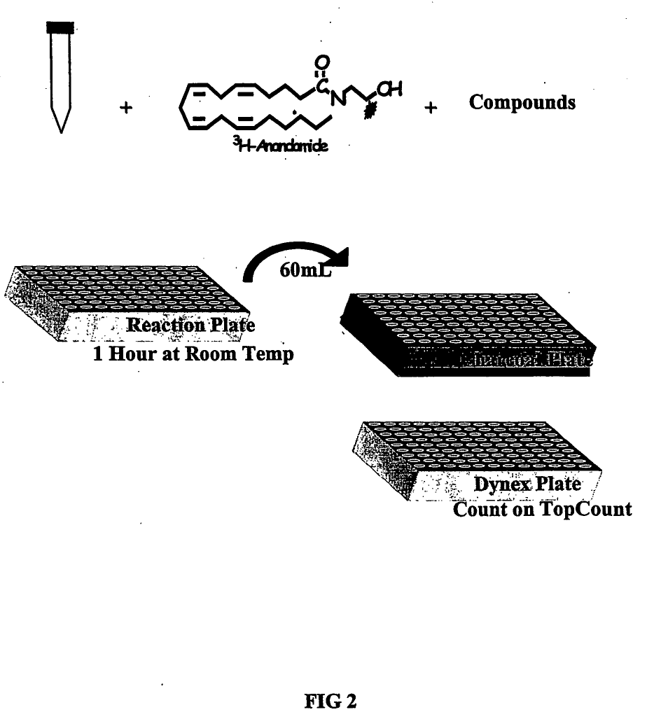

Uncleaved ³H-anandamide, as well as the unlabeled arachidonic acid, is absorbed by the charcoal. In contrast, the labeled ³H-ethanolamine flows through the charcoal mini-columns into 96 well counting plates when a vacuum or centrifugation is applied. The 96 well plates can then be read on a Hewlett Packard TopCount (FIG. 2.)

Charcoal Selectively Binds Anandamide and Arachidonic Acid, but Not Ethanolamine:

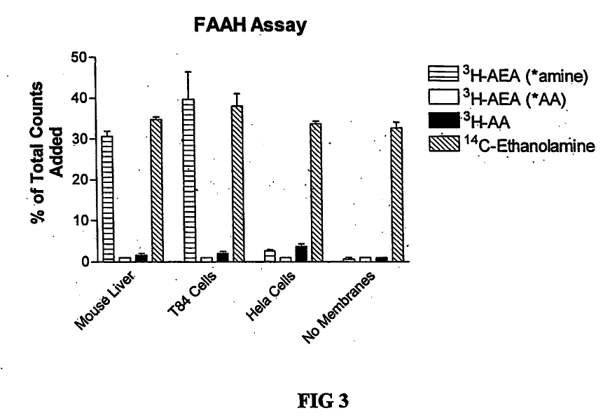

Four different radioactive tracers were used to explore the binding characteristics of the charcoal used in the assay: two tritiated forms of anandamide (labeled either on the ethanolamine moiety; anandamide [1-³H-ethanolamine] or on the arachidonic acid moiety; arachidonic acid [5,6,8,9,11,12,14,15-³H (N)]), as well as tritiated arachidonic acid, and ¹⁴C-labeled ethanolamine. Specific amounts of these tracers were incubated with membrane preparations, added to the pre-washed charcoal and recovered by centrifugation, as described in the methods. The recovered radioactivity was counted and expressed as percentage of the amount added. As shown in FIG. 3, when no membranes were present, i.e., there was no FAAH-mediated conversion of anandamide to arachidonic acid plus ethanolamine, neither of the two labeled forms of anandamide could be detected in the flow-through, indicating that the tracers were bound to the charcoal. Tritiated arachidonic acid was also absorbed onto the charcoal and could not be detected in the flowthrough. Of the four tracers used, only the ¹⁴C-ethanolamine could be recovered.

It is known that HeLa cells, a human carcinoma cell line, do not express FAAH (Ueda et al. (2000) Chem. Phys. Lipids. 108:107-121). Therefore, this cell line was used as a negative control, and to investigate the possible contribution of non-FAAH enzymes to the hydrolysis of anandamide. There was essentially no recovery of radioactivity after incubation of ³H-anandamide with HeLa cell membranes, confirming the absence of FAAH (FIG. 3).

When the tracers were incubated with membranes prepared from mouse liver or T84 cells, good recovery of radioactivity was found for the anandamide labeled on the N-terminus of the amide (³H-ethanolamine), but not for the anandamide carrying the tritium label on the hydrocarbon chain of the fatty acid (³H-AA). All experiments consistently showed that anandamide and arachidonic acid, in contrast to ethanolamine, were absorbed onto the charcoal column. Therefore, when radiolabeled anandamide is incubated with a cell lysate containing FAAH the radioactivity recovered in the flow-through must be attributed to the radiolabeled ethanolamine. This experiment indicates that it is possible to separate anandamide and ethanolamine using charcoal. Other products, such as SAX and C18 resin were also tested, but were less effective (not shown).

Characterization of the FAAH Assay:

FAAH activity is found in a large variety of cells. In rodents, the liver, followed by the brain, seems to have the highest expression of FAAH, whereas in humans, the expression is highest in the pancreas and brain, but lower in the liver (Ueda et al. (2000) Chem. Phys. Lipids. 108:107-121). The expression of human FAAH mRNA in T84 cells was found by expression profiling using DNA microarrays (not shown) and confirmed by subsequent experiments.

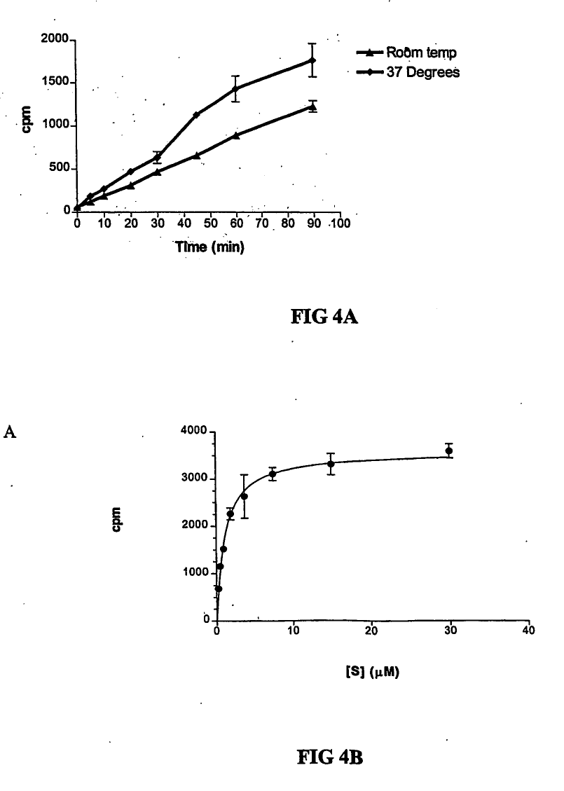

The signal output of the assay was determined to be linear in the range of 0.42-3.5 μg/well T84 membrane. Since an embodiment of the invention is a HTS-compatible assay, it was verified that the reaction was linear over more than 1 hour at the amount of protein used. As shown in FIG. 4A, the rate of the reaction at room temperature was linear over a period of 90 minutes when 3.5 μg/well protein was used. This experiment was performed both at room temperature and at 37° C., with the enzyme being slightly more active at the higher temperature, as expected. For the performance of HTS assays, an incubation period of 1 hour at room temperature and a protein amount of 3.5 μg/well was chosen based on these results.

Kinetic Analysis of FAAH Activity:

Kinetic analyses of the hydrolysis of anandamide by FAAH in T84 human colorectal carcinoma cells demonstrated a Kₘ of 1.1±0.17 μM. (FIG. 4B). This is close to the value that has been reported for human brain (2 μM, see Maccarrone et al. (Maccarrone et al. (1999) Anal. Biochem. 267:314-318)). Overall, the reported Kₘ values in the literature for FAAH reactions range widely, from 0.8 to 80 μM, depending on the tissue, species and method used (Fowler et al. (2001) Biochem. Pharmacol. 62:517-526). For instance, Desarnaud et al. (Desarnaud et al. (1995) J. Biol. Chem. 270, 6030-6035) found a Kₘ of 12.7 μM using rat brain microsomes. In the N18 mouse neuroblastoma cell line, the Kₘ was determined to be 9.0 μM (Maurelli et al. (1995) FEBS Lett. 377:82-86). Apart from species differences, the variability in these values has been ascribed to the observation that both the substrate and product of this enzyme can form micelles, which may affect the enzyme activity (Fowler et al. (2001) Biochem. Pharmacol. 62:517-526).

Validation of the FAAH Assay Using Reference Compounds:

Several of the FAAH inhibitors that have been described in the literature were tested in the assay to validate its ability to identify inhibitors. Of the inhibitors that are commercially available, methyl arachidonyl fluorophosphate (MAFP), which is also an inhibitor of cytosolic phospholipase A₂, is the most potent, with reported IC₅₀ values of 1-3 nM (Ueda et al. (2000) Chem. Phys. Lipids 108, 107-121; Deutsch et al. (1997) Biochem. Pharmacol. 53, 255-260). Its potency as an inhibitor of FAAH was confirmed with the determination of an IC₅₀ value of 0.8 nM.

Oleyl trifluoromethyl ketone, a transition-state inhibitor; has also been reported to be a potent inhibitor of FAAH, with an IC₅₀ of 73.3 nM in the studies here presented, it was in the same range as the IC₅₀ values of 28-41 nM found by Tiger et al. (2000; Biochem. Pharmacol. 59, 647-653).

Phenylmethanesulfonyl fluoride, an inhibitor of serine proteases, was shown to be an inhibitor of FAAH although with a low potency. Its pIC50 for the inhibition of rat brain FAAH in another group was between 5.92 and 4.16, depending on the pH (Holt et al. (2001) Br. J. Pharmacol. 133, 513-520). At a concentration of 100 μM, it abolished anandamide hydrolysis in N18 mouse neuroblastoma cells (Maurelli et al. (1995) FEBS Lett. 377, 82-86). It was used at a concentration of 1.5 mM by Deutsch et al. (1993; Biochem. Pharmacol. 46, 791-796) to block FAAH activity in neuroblastoma and glial cells. At 25 μM, it inhibited the hydrolysis of rat brain microsomal anandamide by 48% (Desarnaud et al. (1995) J. Biol. Chem., 270, 6030-6035). The IC₅₀ value (16 μM obtained in this work thus corresponds well to the numbers reported in the literature.

Finally, product inhibition was tested by using the FAAH products oleic acid and arachidonic acid. Maurelli et al. (1995; FEBS Lett. 377, 82-86) found that 100 μM anandamide abolished the activity of FAAH. In this work, these compounds inhibited FAAH activity with IC₅₀ values in the low micromolar range (Table 1).

These results confirm that the assay provides a robust method for the evaluation of FAAH activity. The assay was successfully validated using reference inhibitors. As the assay may be performed simultaneously in multiple reactions, such as in a 96 well plate, for example, the assay is adaptable for high-throughput screening of compound collections as well as natural product or combinatorial libraries.

TABLE 1
Activity of known FAAH inhibitors in T84 membranes

| COMPOUND | IC₅₀ (nM) | Literature (ref) |
|---|---|---|
| Methyl arachidonyl | 0.8 ± 0.7 | 2.5 ⁽¹⁾ |
| fluorophosphate | | 1-3 ⁽²⁾ |
| Oleyl trifluoromethyl ketone | 73.3 ± 19.8 | 39 ⁽³⁾ |
| | | 24-41 ⁽⁴⁾ |
| Phenylmethylsulfonyl fluoride | 15978 ± 8863 | 1200-69000 ⁽⁵⁾ |
| Arachidonic acid | 931.4 ± 255.4 | — |
| Oleic acid | 1936 ± 401 | — |

The average ± S.E. of values obtained in 2-5 experiments, each done in triplicate, is given. The numbers in the column on the right refer to literature data.
References:
⁽¹⁾ Deutsch et al. Biochem. Pharmacol. 1997, 53: 255-260.
⁽²⁾ Ueda et al. Chem. Phys. Lipids 2000, 108: 107-121.
⁽³⁾ Fowler et al. Br. J. Pharmacol. 2000, 131: 498-504.
⁽⁴⁾ Tiger et al. Biochem. Pharmacol. 2000, 59: 647-653.
⁽⁵⁾ Holt et al. Br. J. Pharmacol. 2001, 133: 513-520.

The foregoing description, examples and accompanying figures generally describe the invention. The description is for purposes of providing illustrations; the present invention is not to be limited by the specific embodiments described herein. One of skill in the art will appreciate that various modifications of the invention may be made in addition to those described herein. Such modifications are intended to fall within the scope of the appended claims.

Pig FAAH Nucleic Acid Sequence (SEQ ID NO:1)
cggtcctcgg tgggagatca tggtgcagga agaactgtgg
gctgcgttct ccggcccctc cggggttgcc ctggcctgct
gcttggtggc agcggccttg gccctgcgtt ggtccagtcg
ccggatggcg cggggcgcgg cggcccgggc gcgacagagg
cagcaagcgg ccctggagac catggacaag gcggcgcagc
gcttccggct ccagaacccc gatctggact cggagatgct
gctggccctg ccactgcctc agctggtaca gaaggtacga
agtggggagc tgtctccaga ggctgtgctc ttttcctacc
tgcaaaaggc ctgggaagtg aacagaggga ccaactgcgt
gaccacctac ctggcagact gtgaggctca gctgtgccag
gcgcccgggc agggcctgct ctacggtgtc cccgtcagcc
tcaaggagtg cttcagctgc aagggccatg actccacgct
gggcttgagc cggaaccagg ggacaccagc agaatgtgac
tgcgtggtgg tgcaggtgct gaaactgcag ggtgctgtgc
ctttcgtgca caccaacgtc ccccagtcca tgttcagcta
tgactgcagt aaccccctct ttggccagac cacgaaccca
tggatgtcgt ccaagagccc gggcggctcc tcgggaggtg
agggggccct cattgctgct ggaggctccc cactgggctt
aggcaccgac atcgggggca gcatccgctt tccctccgcc
ttctgtggca tctgcggcat caaacccacg gggaatcgca
ccagcaagag tggtctgaag ggctctgtct atggacaggt
agcagtgcag ctctcagtgg gccccatggc gcgggacgtg
gagagcctgg ccctgtgcct gcgtgcgctg ctgtgcgaag
acatgttccg cctggacccc acggtgcctc ccctgccctt
caacgaggag gtctacgcaa gctctcggcc cctgcgtgtc
gggtattatg agaccgacaa ctacaccatg cccacgccgg
ccatgaggcg ggccctgctg gagaccaagc ggagccttga
ggctgcgggc cacacgctga ttcccttcct gccggccaac
ataccccacg ctctggaggc cctgtcaacg ggcgggctct
tcagtgatgg tgggaagagg ttgctacaga acttcgaagg
cgattacgtg gactcctgct taggggacct gatctcaatt
ctgaggctgc ccaaatggct taaaggactg ctggctttca
tgctgaggcc tctgctccca aggttggcag gctttctcag
cagcctgagg cctcggtcgg ctggaaagct ctgggaactg
cagcacgaga ttgagatgta ccgtcactcc gtgattgccc
agtggcgagc gctggacctg gatgtggtgc taacccccat
gctgagccct gccctagact tgaatgcccc aggcaaggcc
acaggggccg tcagctacac gctgctctac aactgcctgg
acttccccgc gggggtggtg cctgtcacca cggtgactgc
cgaggacgag gcccagatgg agcattacaa gggctacttt
ggggacattt gggacaaggt ggtgcagaag gccatgaaga
ggagcgtggg gctgcctgtg gccgtgcagt gtgtggctct
gccctggcag gaggagctgt gtttgcggtt catgcgggag
gtggagcgac tgatggctcc tgggcggcag ccctcctgac
cgctgcccgc ccggcccccc aggacctgag acccactgga
tccgcgccca gcggagtcag gacacaactg ccaccgtgca
agaaaatgtt caacctcagg cagaggcttc ccggtctctc
cccctcgccc ctgccagaag cccagaacca ctgagtctgg
accttgctct tcccgtggtc cctgctctgc cctgaccccg
ccaatgtggc agctagtggg tatgacatgg caaaggcccc
ccaaccgtca aaaaccggtt cctggtctcc atactttctg
gcagtcgttg ttagggcagt gggggttgga gacctgacct
tctggaaccc gactccagcc atgtccgtct cgtgctgcag
aagcttctct ggtcctcgtc actcacgggc agacaccggc
ttctccgagt gggccttgca gcccaggact tcaccccgcc
gcccccagcc taagccctac tttgcgaggc attgtcttct
ctcctgccct ctgctgaggg tgccctttct gctcctctac
cattaaatcc tttgaggccc
Pig FAAH Amino Acid Sequence (SEQ ID NO:2)
MVQEELWAAF SGPSGVALAC CLVAAALALR WSSRRMARGA
AARARQRQQA ALETMDKAAQ RFRLQNPDLD SEMLLALPLP
QLVQKVRSGE LSPEAVLFSY LQKAWEVNRG TNCVTTYLAD
CEAQLCQAPG QGLLYGVPVS LKECFSCKGH DSTLGLSRNQ
GTPAECDCVV VQVLKLQGAV PFVHTNVPQS MFSYDCSNPL
FGQTTNPWMS SKSPGGSSGG EGALIAAGGS PLGLGTDIGG
SIRFPSAFCG ICGIKPTGNR ISKSGLKGSV YGQVAVQLSV
GPMARDVESL ALCLRALLCE DMFRLDPTVP PLPFNEEVYA
SSRPLRVGYY ETDNYTMPTP AMRRALLETK RSLEAAGHTL
IPFLPANIPH ALEALSTGGL FSDGGKRLLQ NFEGDYVDSC
LGDLISILRL PKWLKGLLAF MLRPLLPRLA GFLSSLRPRS
AGKLWELQHE IEMYRHSVIA QWRALDLDVV LTPMLSPALD
LNAPGKATGA VSYTLLYNCL DFPAGVVPVT TVTAEDEAQM
EHYKGYFGDI WDKVVQKAMK RSVGLPVAVQ CVALPWQEEL
CLRFMREVER LMAPGRQPS
Mouse FAAH Nucleic Acid Sequence (SEQ ID NO:3)
atggtgctga gcgaagtgtg gaccgcgctg tctggactct
ccggggtttg cctagcctgc agcttgctgt cggcggcggt
ggtcctgcga tggaccagga gccagaccgc ccggggcgcg
gtgaccaggg cgcggcagaa gcagcgagcc ggcctggaga
ccatggacaa ggcggtgcag cgcttccggc tgcagaatcc
tgacctggat tcagaggcct tgctggctct gcccctgctc
caactggtac agaagttaca gagtggggaa ctgtccccag
aagctgtgct ctttacctac ctgggaaagg cctgggaagt
gaacaaaggg accaactgtg tgacctccta tctgactgac
tgtgagactc agctgtccca ggccccacgg cagggcctgc
tctatggcgt ccccgtgagc ctcaaggaat gcttcagcta
caagggccat gcttccacac tgggcttaag tttgaacgag
ggtgtgacat cggagagtga ctgtgtggtg gtgcaggtac
tgaagctgca gggagctgtg ccctttgtgc acaccaacgt
cccccagtcc atgctaagct atgactgcag taaccccctc
tttggccaga ccatgaaccc gtggaagccc tccaagagtc
caggaggttc ctcagggggt gagggggctc tcattggatc
tggaggctcc cctctgggtt taggcactga catcggcggc
agcatccggt tcccttctgc cttctgtggc atctgtggcc
tcaagcctac tgggaaccgc ctcagcaaga gtggcctgaa
gagctgtgtt tatggacaga cagcagtgca gctttctgtt
ggccccatgg cacgggatgt ggatagcctg gcattgtgca
tgaaagccct actttgtgag gatttgttcc gcttggactc
caccatcccc cccttgccct tcagggagga gatctacaga
agttctcgac cccttcgtgt gggatactat gaaactgaca
actacaccat gcccactcca gccatgagga gggctgtgat
ggagaccaag cagagtctcg aggctgctgg ccacacgctg
gtccccttct taccaaacaa cataccttat gccctggagg
tcctgtcggc aggtgggctg ttcagtgatg gtggctgctc
ttttctccaa aacttcaaag gcgactttgt ggatccctgc
ttgggggacc tggtcttagt gctgaagctg cccaggtggt
ttaaaaaact gctgagcttc ctgctgaagc ctctgtttcc
tcggctggca gcctttctca acagtatgtg tcctcggtca
gccgaaaagc tgtgggaact gcagcatgag attgagatgt
atcgccagtc cgtcattgcc cagtggaagg caatgaactt
ggacgtggtg ctaaccccca tgctgggtcc tgctctggat
ttgaacacac cgggcagagc cacaggggct atcagctaca
ctgttctcta taactgcctg gacttccctg cgggggtggt
gcctgtcacc actgtgaccg ctgaggacga tgcccagatg
gaacactaca aaggctactt tggggatatg tgggacaaca
ttctgaagaa gggcatgaaa aagggtatag gcctgcctgt
ggctgtgcag tgcgtggctc tgccctggca ggaagagctg
tgtctgcggt tcatgcggga ggtggaacgg ctgatgaccc
ctgaaaagcg gccatcttga
Mouse FAAH Amino Acid Sequence (SEQ ID NO:4)
MVLSEVWTAL SGLSGVCLAC SLLSAAVVLR WTRSQTARGA
VTRARQKQRA GLETMDKAVQ RFRLQNPDLD SEALLALPLL
QLVQKLQSGE LSPEAVLFTY LGKAWEVNKG TNCVTSYLTD
CETQLSQAPR QGLLYGVPVS LKECFSYKGH ASTLGLSLNE
GVTSESDCVV VQVLKLQGAV PFVHTNVPQS MLSYDCSNPL
FGQTMNPWKP SKSPGGSSGG EGALIGSGGS PLGLGTDIGG
SIRFPSAFCG ICGLKPTGNR LSKSGLKSCV YGQTAVQLSV
GPMARDVDSL ALCMKALLCE DLFRLDSTIP PLPFREEIYR
SSRPLRVGYY ETDNYTMPTP AMRRAVMETK QSLEAAGHTL
VPFLPNNIPY ALEVLSAGGL FSDGGCSFLQ NFKGDFVDPC
LGGLVLVLKL PRWFKKLLSF LLKPLFPRLA AFLNSMCPRS
AEKLWELQHE IEMYRQSVIA QWKAMNLDVV LTPMLGPALD
LNTPGRATGA ISYTVLYNCL DFPAGVVPVT TVTAEDDAQM
EHYKGYFGDM WDNILKKGMK KGIGLPVAVQ CVALPWQEEL
CLRFMREVER LMTPEKRPS
Rat FAAH Nucleic Acid Sequence (SEQ ID NO:5)
ggtttgtgcg agccgagttc tctcgggtgg cggtcggctg
caggagatca tggtgctgag cgaagtgtgg accacgctgt
ctggggtctc cggggtttgc ctagcctgca gcttgttgtc
ggcggcggtg gtcctgcgat ggaccgggcg ccagaaggcc
cggggcgcgg cgaccagggc gcggcagaag cagcgagcca
gcctggagac catggacaag gcggtgcagc gcttccggct
gcagaatcct gacctggact cggaggcctt gctgaccctg
cccctactcc aactggtaca gaagttacag agtggagagc
tgtccccaga ggctgtgttc tttacttacc tgggaaaggc
ctgggaagtg aacaaaggga ccaactgcgt gacctcctat
ctgaccgact gtgagactca gctgtcccag gccccacggc
agggcctgct ctatggtgtc cctgtgagcc tcaaggaatg
cttcagctac aagggccacg actccacact gggcttgagc
ctgaatgagg gcatgccatc ggaatctgac tgtgtggtgg
tgcaagtgtt gaagctgcag ggagctgtgc cctttgtgca
taccaatgtc ccccagtcca tgttaagctt tgactgcagt
aaccctctct ttggccagac catgaaccca tggaagtcct
ccaagagccc aggaggttcc tcagggggtg agggggctct
cattggatct ggaggttccc ctctgggttt aggcactgac
attggcggca gcatccggtt cccttctgcc ttctgcggca
tctgtggcct caagcctact ggcaaccgcc tcagcaagag
tggcctgaag ggctgtgtct atggacagac ggcagtgcag
ctttctcttg gccccatggc ccgggatgtg gagagcctgg
cgctatgcct gaaagctcta ctgtgtgagc acttgttcac
cttggaccct accgtgcctc ccttgccctt cagagaggag
gtctatagaa gttctagacc cctgcgtgtg gggtactatg
agactgacaa ctataccatg cccagcccag ctatgaggag
ggctctgata gagaccaagc agagacttga ggctgctggc
cacacgctga ttcccttctt acccaacaac ataccctacg
ccctggaggt cctgtctgcg ggcggcctgt tcagtgacgg
tggccgcagt tttctccaaa acttcaaagg tgactttgtg
gatccctgct tgggagacct gatcttaatt ctgaggctgc
ccagctggtt taaaagactg ctgagcctcc tgctgaagcc
tctgtttcct cggctggcag cctttctcaa cagtatgcgt
cctcggtcag ctgaaaagct gtggaaactg cagcatgaga
ttgagatgta tcgccagtct gtgattgccc agtggaaagc
gatgaacttg gatgtgctgc tgacccccat gttgggccct
gctctggatt tgaacacacc gggcagagcc acaggggcta
tcagctacac cgttctctac aactgcctgg acttccctgc
gggggtggtg cctgtcacca ctgtgaccgc cgaggacgat
gcccagatgg aactctacaa aggctacttt ggggatatct
gggacatcat cctgaagaag gccatgaaaa atagtgtcgg
tctgcctgtg gctgtgcagt gcgtggctct gccctggcag
gaagagctgt gtctgaggtt catgcgggag gtggaacagc
tgatgacccc tcaaaagcag ccatcgtgag ggtcgttcat
ccgccagctc tggaggacct aaggcccatg cgctgtgcac
tgtagcccca tgtattcagg agccaccacc cacgagggaa
cgcccagcac agggaagagg tgtctacctg ccctcccctg
gactcctgca gccacaacca agtctggacc ttcctccccg
ttatggtcta ctttccatcc tgattccctg ctttttatgg
cagccagcag gaatgacgtg ggccaaggat caccaacatt
caaaaacaat gcgtttatct attttctggg tatctccatt
agggccctgg gaaccagagt gctgggaagg ctgtccagac
cctccagagc tggctgtaac cacatcactc tcctgctcca
aagcctccct agttctgtca cccacaagat agacacaggg
acatgtcctt ggcacttgac tcctgtcctt cctttcttat
tcagattgac cccagccttg atggaccctg cccctgtact
tccttcctca gtccacctct ctgccgacac gcccttttta
tggctcctct atttgttgtg gagacaaggt ttctctcagt
agccctggct gtccaggacc tcactctgta gatgaggctg
gctttcaact cacaaggctg cctgcctggg tgctgggatt
aaaggcgtat gccaccacaa agaaaaaaaa aa
Rat FAAH Amino Acid Sequence (SEQ ID NO:6)
MVLSEVWTTL SGVSGVCLAC SLLSAAVVLR WTGRQKARGA
ATRARQKQRA SLETMDKAVQ RFRLQNPDLD SEALLTLPLL
QLVQKLQSGE LSPEAVFFTY LGKAWEVNKG TNCVTSYLTD
CETQLSQAPR QGLLYGVPVS LKECFSYKGH DSTLGLSLNE
GMPSESDCVV VQVLKLQGAV PFVHTNVPQS MLSFDCSNPL
FGQTMNPWKS SKSPGGSSGG EGALIGSGGS PLGLGTDIGG
SIRFPSAFCG ICGLKPTGNR LSKSGLKGCV YGQTAVQLSL
GPMARDVESL ALCLKALLCE HLFTLDPTVP PLPFREEVYR
SSRPLRVGYY ETDNYTMPSP AMRRALIETK QRLEAAGHTL
IPFLPNNIPY ALEVLSAGGL FSDGGRSFLQ NFKGDFVDPC
LGDLILILRL PSWFKRLLSL LLKPLFPRLA AFLNSMRPRS
AEKLWKLQHE IEMYRQSVIA QWKAMNLDVL LTPMLGPALD
LNTPGRATGA ISYTVLYNCL DFPAGVVPVT TVTAEDDAQM
ELYKGYFGDI WDIILKKAMK NSVGLPVAVQ CVALPWQEEL
CLRFMREVEQ LMTPQKQPS
Human FAAH Nucleic Acid Sequence (SEQ ID NO:7)
tgccgggcgg taggcagcag caggctgaag ggatcatggt
gcagtacgag ctgtgggccg cgctgcctgg cgcctccggg
gtcgccctgg cctgctgctt cgtggcggcg gccgtggccc
tgcgctggtc cgggcgccgg acggcgcggg gcgcggtggt
ccgggcgcga cagaagcagc gagcgggcct ggagaacatg
gacagggcgg cgcagcgctt ccggctccag aacccagacc
tggactcaga ggcgctgcta gccctgcccc tgcctcagct
ggtgcagaag ttacacagta gagagctggc ccctgaggcc
gtgctcttca cctatgtggg aaaggcctgg gaagtgaaca
aagggaccaa ctgtgtgacc tcctatctgg ctgactgtga
gactcagctg tctcaggccc caaggcaggg cctgctctat
ggcgtccctg tgagcctcaa ggagtgcttc acctacaagg
gccaggactc cacgctgggc ttgagcctga atgaaggggt
gccggcggag tgcgacagcg tagtggtgca tgtgctgaag
ctgcagggtg ccgtgccctt cgtgcacacc aatgttccac
agtccatgtt cagctatgac tgcagtaacc ccctctttgg
ccagaccgtg aacccatgga agtcctccaa aagcccaggg
ggctcctcag ggggtgaagg ggccctcatc gggtctggag
gctcccccct gggcttaggc actgatatcg gaggcagcat
ccgcttcccc tcctccttct gcggcatctg cggcctcaag
cccacaggga accgcctcag caagagtggc ctgaagggct
gtgtctatgg acaggaggca gtgcgtctct ccgtgggccc
catggcccgg gacgtggaga gcctggcact gtgcctgcga
gccctgctgt gcgaggacat gttccgcttg gaccccactg
tgcctccctt gcccttcaga gaagaggtct acaccagctc
tcagcccctg cgtgtggggt actatgagac tgacaactat
accatgccct ccccggccat gaggcgggcc gtgctggaga
ccaaacagag ccttgaggct gcggggcaca cgctggttcc
cttcttgcca agcaacatac cccatgctct ggagaccctg
tcaacaggtg ggctcttcag tgatggtggc cacaccttcc
tacagaactt caaaggtgat ttcgtggacc cctgcctggg
ggacctggtc tcaattctga agcttcccca atggcttaaa
ggactgctgg ccttcctggt gaagcctctg ctgccaaggc
tgtcagcttt cctcagcaac atgaagtctc gttcggctgg
aaaactctgg gaactgcagc acgagatcga ggtgtaccgc
aaaaccgtga ttgcccagtg gagggcgctg gacctggatg
tggtgctgac ccccatgctg gcccctgctc tggacttgaa
tgccccaggc agggccacag gggccgtcag ctacactatg
ctgtacaact gcctggactt ccctgcaggg gtggtgcctg
tcaccacggt gactgctgag gacgaggccc agatggaaca
ttacaggggc tactttgggg atatctggga caagatgctg
cagaagggca tgaagaagag tgtggggctg ccggtggccg
tgcagtgtgt ggctctgccc tggcaagaag agttgtgtct
gcggttcatg cgggaggtgg agcgactgat gacccctgaa
aagcagtcat cctgatggct ctggctccag aggacctgag
actcacactc tctgcagccc agcctagtca gggcacagct
gccctgctgc cacagcaagg aaatgtcctg catggggcag
aggcttccgt gtcctctccc ccaaccccct gcaagaagcg
ccgactccct gagtctggac ctccatccct gctctggtcc
cctctcttcg tcctgatccc tccaccccca tgtggcagcc
catgggtatg acataggcca aggcccaact aacagtcaag
aaacaaaaaa aaaaaaaaaa aaa
Human FAAH Amino Acid Sequence (SEQ ID NO:8)
MVQYELWAAL PGASGVALAC CFVAAAVALR WSGRRTARGA
VVRARQKQRA GLENMDRAAQ RFRLQNPDLD SEALLALPLP
QLVQKLHSRE LAPEAVLFTY VGKAWEVNKG TNCVTSYLAD
CETQLSQAPR QGLLYGVPVS LKECFTYKGQ DSTLGLSLNE
GVPAECDSVV VHVLKLQGAV PFVHTNVPQS MFSYDCSNPL
FGQTVNPWKS SKSPGGSSGG EGALIGSGGS PLGLGTDIGG
SIRFPSSFCG ICGLKPTGNR LSKSGLKGCV YGQEAVRLSV
GPMARDVESL ALCLRALLCE DMFRLDPTVP PLPFREEVYT
SSQPLRVGYY ETDNYTMPSP AMRRAVLETK QSLEAAGHTL
VPFLPSNIPH ALETLSTGGL FSDGGHTFLQ NFKGDFVDPC
LGDLVSILKL PQWLKGLLAF LVKPLLPRLS AFLSNMKSRS
AGKLWELQHE IEVYRKTVIA QWRALDLDVV LTPMLAPALD
LNAPGRATGA VSYTMLYNCL DFPAGVVPVT TVTAEDEAQM
EHYRGYFGDI WDKMLQKGMK KSVGLPVAVQ CVALPWQEEL
CLRFMREVER LMTPEKQSS

What is claimed is:

1. A method of assaying the activity of a fatty acid amide hydrolase comprising the steps of:
combining a sample suspected of containing a fatty acid amide hydrolase, with a labeled substrate of the fatty acid amide hydrolase, to form a reaction mixture;
incubating the reaction mixture under conditions sufficient to allow the fatty acid amide hydrolase to hydrolyze the labeled substrate, thereby forming at least one labeled hydrolysis product;
contacting the incubated reaction mixture with a selective binding material; wherein the selective binding material binds either the labeled substrate or a labeled hydrolysis product, but not both, thereby forming a bound labeled complex;
separating the bound labeled complex from the incubated reaction mixture; and
determining an amount of labeled substrate hydrolyzed, or labeled hydrolysis product formed, thereby indicating the fatty acid amide hydrolase activity of the sample.

2. The method of claim 1 wherein the sample comprises biological membranes, lipid bilayers, or micelles.

3. The method of claim 1 wherein the substrate is an endocannabinoid, a fatty acid ethanolamide, a fatty acid primary amide, an endocannabinoid analog, a fatty acid ethanolamide analog, or a fatty acid primary amide analog.

4. The method of claim 1 wherein the substrate is anandamide.

5. The method of claim 1 wherein the substrate is oleamide.

6. The method of claim 1 wherein the substrate is 2-arachidonoylglycerol.

7. The method of claim 1 wherein the substrate is labeled with a radioisotope.

8. The method of claim 7 wherein the radioisotope is ³H or ¹⁴C.

9. The method of claim 1 wherein the substrate is labeled with a fluorescent label.

10. The method of claim 1 wherein the selective binding material comprises carbon.

11. The method of claim 10 wherein the selective binding material is activated charcoal.

12. The method of claim 11 wherein the activated charcoal comprises a filter.

13. The method of claim 1 wherein the selective binding material binds the labeled substrate but not the labeled product.

14. The method of claim 1 wherein the separating step comprises filtration, gravity settling or centrifugation.

15. The method of claim 1 wherein the determining step is performed via liquid scintillation counting or by measurement of fluorescence energy.

16. The method of claim 1 conducted in a multiwell plate.

17. The method of claim 1 comprising at least a portion of a high throughput screening program.

18. The method of claim 1 wherein the method is conducted in conjunction with a drug discovery effort.

19. A method of identifying a compound that modulates the activity of a fatty acid amide hydrolase comprising the steps of:
comparing the activity of a fatty acid amide hydrolase as assayed by the method of claim 1, in the presence and in the absence of a test compound added to the reaction mixture;
wherein a change in the activity of the fatty acid amide hydrolase indicates that the test compound modulates the activity of the fatty acid amide hydrolase.

20. The method of claim 19 wherein the test compound is selected from a library of compounds.

21. The method of claim 19 wherein the test compound inhibits the activity of the fatty acid amide hydrolase activity.

22. The method of claim 21 wherein said fatty acid amide hydrolase activity is inhibited at least about 5%.

23. The method of claim 21 wherein said fatty acid amide hydrolase activity is inhibited at least about 20%.

24. The method of claim 21 wherein said fatty acid amide hydrolase activity is inhibited at least about 50%.

25. The method of claim 21 wherein said fatty acid amide hydrolase activity is inhibited at least about 80%.

26. The method of claim 21 wherein said fatty acid amide hydrolase activity is inhibited at least about 95% or more.

27. The method of claim 21 wherein said test compound increases said fatty acid amide hydrolase activity.

28. The method of claim 27 wherein said fatty acid amide hydrolase activity is increased at least about 5%.

29. The method of claim 27 wherein said fatty acid amide hydrolase activity is increased at least about 30%.

30. The method of claim 27 wherein said fatty acid amide hydrolase activity is increased at least about 50%.

31. The method of claim 27 wherein said fatty acid amide hydrolase activity is increased at least about 70%.

32. The method of claim 27 wherein said fatty acid amide hydrolase activity is increased at least about 100%.

33. The method of claim 27 wherein said fatty acid amide hydrolase activity is increased between about two-fold to about ten-fold.

34. The method of claim 19, which comprises the use of a multi-well plate.

35. The method of claim 19 conducted in a multiwell plate.

36. The method of claim 19 comprising at least a portion of a high throughput screening.

37. The method of claim 19 wherein the method is conducted in conjunction with a drug discovery effort.

38. The method of claim 1 wherein said fatty acid amide hydrolase is a mammalian fatty acid amide hydrolase.

39. The method of claim 38 wherein said fatty acid amide hydrolase is a porcine fatty acid amide hydrolase.

40. The method of claim 38 wherein said fatty acid amide hydrolase is a rodent fatty acid amide hydrolase.

41. The method of claim 38 wherein said fatty acid amide hydrolase is a murine fatty acid amide hydrolase.

42. The method of claim 41 wherein said fatty acid amide hydrolase is a rat fatty acid amide hydrolase.

43. The method of claim 41 wherein said fatty acid amide hydrolase is a mouse fatty acid amide hydrolase.

44. The method of claim 38 wherein said fatty acid amide hydrolase is a human fatty acid amide hydrolase.

45. A method for determining altered fatly acid amide hydrolase activity in a patient comprising:
obtaining a sample containing cells from the patient;
lysing the cells to form a cell lysate;
combining the cell lysate with a labeled substrate of fatty acid amide hydrolase, to form a reaction mixture;
incubating the reaction mixture under conditions sufficient to allow a fatty acid amide hydrolase present in the cell lysate to hydrolyze the labeled substrate, thereby forming at least one labeled hydrolysis product;
contacting the incubated reaction mixture with a selective binding material; wherein the selective binding material binds either the labeled substrate or a labeled hydrolysis product, but not both, thereby forming a bound labeled complex;
separating the bound labeled complex from the incubated reaction mixture;
determining an amount of labeled substrate hydrolyzed, or labeled hydrolysis product formed, thereby indicating the fatty acid amide hydrolase activity of the sample; and
comparing the activity of the sample from the patient with the activity of a to a predetermined value for activity, to determine if the patient has altered fatty acid amide hydrolase activity relative to the predetermined value for activity.

46. The method of claim 45 wherein said patient is female.

47. The method of claim 46 wherein the female is pregnant or is seeking fertility treatment.

48. The method of claim 45 wherein the sample comprises blood, tissue or body fluid.

49. The method of claim 46 wherein the sample comprises lymphocytes.

50. The method of claim 45 wherein the cells are homogenized.

51. The method of claim 45 wherein the fatty acid amide hydrolase activity present in the cell lysate is partially or substantially purified from the sample.

52. The method of claim 45 wherein the predetermined determined value is from a control assay, a prior or subsequent sample from the patient, a sample from a normal individual, a sample from another patient, a standard FAAH, or a predetermined value.


SEQUENCE LISTING (8 sequences)

SEQ ID NO: 1 - DNA, 2300 bp, Sus scrofa
cggtcctcggtgggagatcatggtgcaggaagaactgtgggctgcgttctccggcccctccggggttgccctggcctgctgcttggtggcagcggccttggccctgcgttggtccagtcgccggatggcgcggggcgcggcggcccgggcgcgacagaggcagcaagcggccctggagaccatggacaaggcggcgcagcgcttccggctccagaaccccgatctggactcggagatgctgctggccctgccactgcctcagctggtacagaaggtacgaagtggggagctgtctccagaggctgtgctcttttcctacctgcaaaaggcctgggaagtgaacagagggaccaactgcgtgaccacctacctggcagactgtgaggctcagctgtgccaggcgcccgggcagggcctgctctacggtgtccccgtcagcctcaaggagtgcttcagctgcaagggccatgactccacgctgggcttgagccggaaccaggggacaccagcagaatgtgactgcgtggtggtgcaggtgctgaaactgcagggtgctgtgcctttcgtgcacaccaacgtcccccagtccatgttcagctatgactgcagtaaccccctctttggccagaccacgaacccatggatgtcgtccaagagcccgggcggctcctcgggaggtgagggggccctcattgctgctggaggctccccactgggcttaggcaccgacatcgggggcagcatccgctttccctccgccttctgtggcatctgcggcatcaaacccacggggaaccgcatcagcaagagtggtctgaagggctctgtctatggacaggtagcagtgcagctctcagtgggccccatggcgcgggacgtggagagcctggccctgtgcctgcgtgcgctgctgtgcgaagacatgttccgcctggaccccacggtgcctcccctgcccttcaacgaggaggtctacgcaagctctcggcccctgcgtgtcgggtattatgagaccgacaactacaccatgcccacgccggccatgaggcgggccctgctggagaccaagcggagccttgaggctgcgggccacacgctgattcccttcctgccggccaacataccccacgctctggaggccctgtcaacgggcgggctcttcagtgatggtgggaagaggttgctacagaacttcgaaggcgattacgtggactcctgcttaggggacctgatctcaattctgaggctgcccaaatggcttaaaggactgctggctttcatgctgaggcctctgctcccaaggttggcaggctttctcagcagcctgaggcctcggtcggctggaaagctctgggaactgcagcacgagattgagatgtaccgtcactccgtgattgcccagtggcgagcgctggacctggatgtggtgctaacccccatgctgagccctgccctagacttgaatgccccaggcaaggccacaggggccgtcagctacacgctgctctacaactgcctggacttccccgcgggggtggtgcctgtcaccacggtgactgccgaggacgaggcccagatggagcattacaagggctactttggggacatttgggacaaggtggtgcagaaggccatgaagaggagcgtggggctgcctgtggccgtgcagtgtgtggctctgccctggcaggaggagctgtgtttgcggttcatgcgggaggtggagcgactgatggctcctgggcggcagccctcctgaccgctgcccgcccggccccccaggacctgagacccactggatccgcgcccagcggagtcaggacacaactgccaccgtgcaagaaaatgttcaacctcaggcagaggcttcccggtctctccccctcgcccctgccagaagcccagaaccactgagtctggaccttgctcttcccgtggtccctgctctgccctgaccccgccaatgtggcagctagtgggtatgacatggcaaaggccccccaaccgtcaaaaaccggttcctggtctccatactttctggcagtcgttgttagggcagtgggggttggagacctgaccttctggaacccgactccagccatgtccgtctcgtgctgcagaagcttctctggtcctcgtcactcacgggcagacaccggcttctccgagtgggccttgcagcccaggacttcaccccgccgcccccagcctaagccctactttgcgaggcattgtcttctctcctgccctctgctgagggtgccctttctgctcctctaccattaaatcctttgaggccc

SEQ ID NO: 2 - protein, 579 aa, Sus scrofa
MVQEELWAAFSGPSGVALACCLVAAALALRWSSRRMARGAAARARQRQQAALETMDKAAQRFRLQNPDLDSEMLLALPLPQLVQKVRSGELSPEAVLFSYLQKAWEVNRGTNCVTTYLADCEAQLCQAPGQGLLYGVPVSLKECFSCKGHDSTLGLSRNQGTPAECDCVVVQVLKLQGAVPFVHTNVPQSMFSYDCSNPLFGQTTNPWMSSKSPGGSSGGEGALIAAGGSPLGLGTDIGGSIRFPSAFCGICGIKPTGNRISKSGLKGSVYGQVAVQLSVGPMARDVESLALCLRALLCEDMFRLDPTVPPLPFNEEVYASSRPLRVGYYETDNYTMPTPAMRRALLETKRSLEAAGHTLIPFLPANIPHALEALSTGGLFSDGGKRLLQNFEGDYVDSCLGDLISILRLPKWLKGLLAFMLRPLLPRLAGFLSSLRPRSAGKLWELQHEIEMYRHSVIAQWRALDLDVVLTPMLSPALDLNAPGKATGAVSYTLLYNCLDFPAGVVPVTTVTAEDEAQMEHYKGYFGDIWDKVVQKAMKRSVGLPVAVQCVALPWQEELCLRFMREVERLMAPGRQPS

SEQ ID NO: 3 - DNA, 1740 bp, Mouse
atggtgctgagcgaagtgtggaccgcgctgtctggactctccggggtttgcctagcctgcagcttgctgtcggcggcggtggtcctgcgatggaccaggagccagaccgcccggggcgcggtgaccagggcgcggcagaagcagcgagccggcctggagaccatggacaaggcggtgcagcgcttccggctgcagaatcctgacctggattcagaggccttgctggctctgcccctgctccaactggtacagaagttacagagtggggaactgtccccagaagctgtgctctttacctacctgggaaaggcctgggaagtgaacaaagggaccaactgtgtgacctcctatctgactgactgtgagactcagctgtcccaggccccacggcagggcctgctctatggcgtccccgtgagcctcaaggaatgcttcagctacaagggccatgcttccacactgggcttaagtttgaacgagggtgtgacatcggagagtgactgtgtggtggtgcaggtactgaagctgcagggagctgtgccctttgtgcacaccaacgtcccccagtccatgctaagctatgactgcagtaaccccctctttggccagaccatgaacccgtggaagccctccaagagtccaggaggttcctcagggggtgagggggctctcattggatctggaggctcccctctgggtttaggcactgacatcggcggcagcatccggttcccttctgccttctgtggcatctgtggcctcaagcctactgggaaccgcctcagcaagagtggcctgaagagctgtgtttatggacagacagcagtgcagctttctgttggccccatggcacgggatgtggatagcctggcattgtgcatgaaagccctactttgtgaggatttgttccgcttggactccaccatcccccccttgcccttcagggaggagatctacagaagttctcgaccccttcgtgtgggatactatgaaactgacaactacaccatgcccactccagccatgaggagggctgtgatggagaccaagcagagtctcgaggctgctggccacacgctggtccccttcttaccaaacaacataccttatgccctggaggtcctgtcggcaggtgggctgttcagtgatggtggctgctcttttctccaaaacttcaaaggcgactttgtggatccctgcttgggggacctggtcttagtgctgaagctgcccaggtggtttaaaaaactgctgagcttcctgctgaagcctctgtttcctcggctggcagcctttctcaacagtatgtgtcctcggtcagccgaaaagctgtgggaactgcagcatgagattgagatgtatcgccagtccgtcattgcccagtggaaggcaatgaacttggacgtggtgctaacccccatgctgggtcctgctctggatttgaacgcaccgggcagagccacaggggctatcagctacactgttctctataactgcctggacttccctgcgggggtggtgcctgtcaccactgtgaccgctgaggacgatgcccagatggaacactacaaaggctactttggggatatgtgggacaacattctgaagaagggcatgaaaaagggtataggcctgcctgtggctgtgcagtgcgtggctctgccctggcaggaagagctgtgtctgcggttcatgcgggaggtggaacggctgatgacccctgaaaagcggccatcttga

SEQ ID NO: 4 - protein, 579 aa, Mouse
MVLSEVWTALSGLSGVCLACSLLSAAVVLRWTRSQTARGAVTRARQKQRAGLETMDKAVQRFRLQNPDLDSEALLALPLLQLVQKLQSGELSPEAVLFTYLGKAWEVNKGTNCVTSYLTDCETQLSQAPRQGLLYGVPVSLKECFSYKGHASTLGLSLNEGVTSESDCVVVQVLKLQGAVPFVHTNVPQSMLSYDCSNPLFGQTMNPWKPSKSPGGSSGGEGALIGSGGSPLGLGTDIGGSIRFPSAFCGICGLKPTGNRLSKSGLKSCVYGQTAVQLSVGPMARDVDSLALCMKALLCEDLFRLDSTIPPLPFREEIYRSSRPLRVGYYETDNYTMPTPAMRRAVMETKQSLEAAGHTLVPFLPNNIPYALEVLSAGGLFSDGGCSFLQNFKGDFVDPCLGDLVLVLKLPRWFKKLLSFLLKPLFPRLAAFLNSMCPRSAEKLWELQHEIEMYRQSVIAQWKAMNLDVVLTPMLGPALDLNAPGRATGAISYTVLYNCLDFPAGVVPVTTVTAEDDAQMEHYKGYFGDMWDNILKKGMKKGIGLPVAVQCVALPWQEELCLRFMREVERLMTPEKRPS

SEQ ID NO: 5 - DNA, 2472 bp, Rattus rattus
ggtttgtgcgagccgagttctctcgggtggcggtcggctgcaggagatcatggtgctgagcgaagtgtggaccacgctgtctggggtctccggggtttgcctagcctgcagcttgttgtcggcggcggtggtcctgcgatggaccgggcgccagaaggcccggggcgcggcgaccagggcgcggcagaagcagcgagccagcctggagaccatggacaaggcggtgcagcgcttccggctgcagaatcctgacctggactcggaggccttgctgaccctgcccctactccaactggtacagaagttacagagtggagagctgtccccagaggctgtgttctttacttacctgggaaaggcctgggaagtgaacaaagggaccaactgcgtgacctcctatctgaccgactgtgagactcagctgtcccaggccccacggcagggcctgctctatggtgtccctgtgagcctcaaggaatgcttcagctacaagggccacgactccacactgggcttgagcctgaatgagggcatgccatcggaatctgactgtgtggtggtgcaagtgttgaagctgcagggagctgtgccctttgtgcataccaatgtcccccagtccatgttaagctttgactgcagtaaccctctctttggccagaccatgaacccatggaagtcctccaagagcccaggaggttcctcagggggtgagggggctctcattggatctggaggttcccctctgggtttaggcactgacattggcggcagcatccggttcccttctgccttctgcggcatctgtggcctcaagcctactggcaaccgcctcagcaagagtggcctgaagggctgtgtctatggacagacggcagtgcagctttctcttggccccatggcccgggatgtggagagcctggcgctatgcctgaaagctctactgtgtgagcacttgttcaccttggaccctaccgtgcctcccttgcccttcagagaggaggtctatagaagttctagacccctgcgtgtggggtactatgagactgacaactataccatgcccagcccagctatgaggagggctctgatagagaccaagcagagacttgaggctgctggccacacgctgattcccttcttacccaacaacataccctacgccctggaggtcctgtctgcgggcggcctgttcagtgacggtggccgcagttttctccaaaacttcaaaggtgactttgtggatccctgcttgggagacctgatcttaattctgaggctgcccagctggtttaaaagactgctgagcctcctgctgaagcctctgtttcctcggctggcagcctttctcaacagtatgcgtcctcggtcagctgaaaagctgtggaaactgcagcatgagattgagatgtatcgccagtctgtgattgcccagtggaaagcgatgaacttggatgtgctgctgacccccatgttgggccctgctctggatttgaacacaccgggcagagccacaggggctatcagctacaccgttctctacaactgcctggacttccctgcgggggtggtgcctgtcaccactgtgaccgccgaggacgatgcccagatggaactctacaaaggctactttggggatatctgggacatcatcctgaagaaggccatgaaaaatagtgtcggtctgcctgtggctgtgcagtgcgtggctctgccctggcaggaagagctgtgtctgaggttcatgcgggaggtggaacagctgatgacccctcaaaagcagccatcgtgagggtcgttcatccgccagctctggaggacctaaggcccatgcgctgtgcactgtagccccatgtattcaggagccaccacccacgagggaacgcccagcacagggaagaggtgtctacctgccctcccctggactcctgcagccacaaccaagtctggaccttcctccccgttatggtctactttccatcctgattccctgctttttatggcagccagcaggaatgacgtgggccaaggatcaccaacattcaaaaacaatgcgtttatctattttctgggtatctccattagggccctgggaaccagagtgctgggaaggctgtccagaccctccagagctggctgtaaccacatcactctcctgctccaaagcctccctagttctgtcacccacaagatagacacagggacatgtccttggcacttgactcctgtccttcctttcttattcagattgaccccagccttgatggaccctgcccctgcacttccttcctcagtccacctctctgccgacacgccctttttatggctcctctatttgttgtggagacaaggtttctctcagtagccctggctgtccaggacctcactctgtagatgaggctggctttcaactcacaaggctgcctgcctgggtgctgggattaaaggcgtatgccaccacaaagaaaaaaaaaa

SEQ ID NO: 6 - protein, 579 aa, Rattus rattus
MVLSEVWTTLSGVSGVCLACSLLSAAVVLRWTGRQKARGAATRARQKQRASLETMDKAVQRFRLQNPDLDSEALLTLPLLQLVQKLQSGELSPEAVFFTYLGKAWEVNKGTNCVTSYLTDCETQLSQAPRQGLLYGVPVSLKECFSYKGHDSTLGLSLNEGMPSESDCVVVQVLKLQGAVPFVHTNVPQSMLSFDCSNPLFGQTMNPWKSSKSPGGSSGGEGALIGSGGSPLGLGTDIGGSIRFPSAFCGICGLKPTGNRLSKSGLKGCVYGQTAVQLSLGPMARDVESLALCLKALLCEHLFTLDPTVPPLPFREEVYRSSRPLRVGYYETDNYTMPSPAMRRALIETKQRLEAAGHTLIPFLPNNIPYALEVLSAGGLFSDGGRSFLQNFKGDFVDPCLGDLILILRLPSWFKRLLSLLLKPLFPRLAAFLNSMRPRSAEKLWKLQHEIEMYRQSVIAQWKAMNLDVLLTPMLGPALDLNTPGRATGAISYTVLYNCLDFPAGVVPVTTVTAEDDAQMELYKGYFGDIWDIILKKAMKNSVGLPVAVQCVALPWQEELCLRFMREVEQLMTPQKQPS

SEQ ID NO: 7 - DNA, 2063 bp, Homo sapiens
tgccgggcggtaggcagcagcaggctgaagggatcatggtgcagtacgagctgtgggccgcgctgcctggcgcctccggggtcgccctggcctgctgcttcgtggcggcggccgtggccctgcgctggtccgggcgccggacggcgcggggcgcggtggtccgggcgcgacagaagcagcgagcgggcctggagaacatggacagggcggcgcagcgcttccggctccagaacccagacctggactcagaggcgctgctagccctgcccctgcctcagctggtgcagaagttacacagtagagagctggcccctgaggccgtgctcttcacctatgtgggaaaggcctgggaagtgaacaaagggaccaactgtgtgacctcctatctggctgactgtgagactcagctgtctcaggccccaaggcagggcctgctctatggcgtccctgtgagcctcaaggagtgcttcacctacaagggccaggactccacgctgggcttgagcctgaatgaaggggtgccggcggagtgcgacagcgtagtggtgcatgtgctgaagctgcagggtgccgtgcccttcgtgcacaccaatgttccacagtccatgttcagctatgactgcagtaaccccctctttggccagaccgtgaacccatggaagtcctccaaaagcccagggggctcctcagggggtgaaggggccctcatcgggtctggaggctcccccctgggcttaggcactgatatcggaggcagcatccgcttcccctcctccttctgcggcatctgcggcctcaagcccacagggaaccgcctcagcaagagtggcctgaagggctgtgtctatggacaggaggcagtgcgtctctccgtgggccccatggcccgggacgtggagagcctggcactgtgcctgcgagccctgctgtgcgaggacatgttccgcttggaccccactgtgcctcccttgcccttcagagaagaggtctacaccagctctcagcccctgcgtgtggggtactatgagactgacaactataccatgccctccccggccatgaggcgggccgtgctggagaccaaacagagccttgaggctgcggggcacacgctggttcccttcttgccaagcaacataccccatgctctggagaccctgtcaacaggtgggctcttcagtgatggtggccacaccttcctacagaacttcaaaggtgatttcgtggacccctgcctgggggacctggtctcaattctgaagcttccccaatggcttaaaggactgctggccttcctggtgaagcctctgctgccaaggctgtcagctttcctcagcaacatgaagtctcgttcggctggaaaactctgggaactgcagcacgagatcgaggtgtaccgcaaaaccgtgattgcccagtggagggcgctggacctggatgtggtgctgacccccatgctggcccctgctctggacttgaatgccccaggcagggccacaggggccgtcagctacactatgctgtacaactgcctggacttccctgcaggggtggtgcctgtcaccacggtgactgctgaggacgaggcccagatggaacattacaggggctactttggggatatctgggacaagatgctgcagaagggcatgaagaagagtgtggggctgccggtggccgtgcagtgtgtggctctgccctggcaagaagagttgtgtctgcggttcatgcgggaggtggagcgactgatgacccctgaaaagcagtcatcctgatggctctggctccagaggacctgagactcacactctctgcagcccagcctagtcagggcacagctgccctgctgccacagcaaggaaatgtcctgcatggggcagaggcttccgtgtcctctcccccaaccccctgcaagaagcgccgactccctgagtctggacctccatccctgctctggtcccctctcttcgtcctgatccctccacccccatgtggcagcccatgggtatgacataggccaaggcccaactaacagtcaagaaacaaaaaaaaaaaaaaaaaaa

SEQ ID NO: 8 - protein, 579 aa, Homo sapiens
MVQYELWAALPGASGVALACCFVAAAVALRWSGRRTARGAVVRARQKQRAGLENMDRAAQRFRLQNPDLDSEALLALPLPQLVQKLHSRELAPEAVLFTYVGKAWEVNKGTNCVTSYLADCETQLSQAPRQGLLYGVPVSLKECFTYKGQDSTLGLSLNEGVPAECDSVVVHVLKLQGAVPFVHTNVPQSMFSYDCSNPLFGQTVNPWKSSKSPGGSSGGEGALIGSGGSPLGLGTDIGGSIRFPSSFCGICGLKPTGNRLSKSGLKGCVYGQEAVRLSVGPMARDVESLALCLRALLCEDMFRLDPTVPPLPFREEVYTSSQPLRVGYYETDNYTMPSPAMRRAVLETKQSLEAAGHTLVPFLPSNIPHALETLSTGGLFSDGGHTFLQNFKGDFVDPCLGDLVSILKLPQWLKGLLAFLVKPLLPRLSAFLSNMKSRSAGKLWELQHEIEVYRKTVIAQWRALDLDVVLTPMLAPALDLNAPGRATGAVSYTMLYNCLDFPAGVVPVTTVTAEDEAQMEHYRGYFGDIWDKMLQKGMKKSVGLPVAVQCVALPWQEELCLRFMREVERLMTPEKQSS